(19) United States
(12) Patent Application Publication
(10) Pub. No.: US 2024/0270745 A1
(43) Pub. Date: Aug. 15, 2024

(54) ISOQUINOLINE ALKALOID COMPOUND, AND PREPARATION METHOD THEREFOR AND USE THEREOF

(71) Applicants: SHANGHAI INSTITUTE OF MATERIA MEDICA, CHINESE ACADEMY OF SCIENCES, Shanghai (CN); LANZHOU INSTITUTE OF CHEMICAL PHYSICS, CHINESE ACADEMY OF SCIENCES, Lanzhou (CN)

(72) Inventors: Zhaobing GAO, Shanghai (CN); Junli YANG, Lanzhou (CN); Xianhua MENG, Lanzhou (CN); Yueming ZHENG, Shanghai (CN); Tian CHAI, Lanzhou (CN); Yin ZHANG, Shanghai (CN)

(21) Appl. No.: 18/569,434

(22) PCT Filed: Jun. 15, 2022
(86) PCT No.: PCT/CN2022/098816

§ 371 Date: Dec. 12, 2023

(30) Foreign Application Priority Data
Jun. 16, 2021 (CN) 202110666488.1

(51) Int. Cl. C07D 471/14; A61K 31/519; A61P 29/00

(52) U.S. Cl. CPC C07D 471/14; A61K 31/519; A61P 29/00

(57) ABSTRACT

Provided in the present invention are an isoquinoline alkaloid compound selected from the group consisting of the compounds represented by the following structural formulas, and a preparation method therefor and the use thereof. The present invention proves for the first time that the isoquinoline alkaloid compound is an inhibitor of the I_{K} potassium current and the background potassium current in the nervous system, the cardiovascular system and the pancreas, especially an inhibitor of the Kv2.1 channel and the TRESK channel, and can be used for treating a disease such as stroke, arrhythmia, atrial fibrillation, diabetes, pain, hypoventilation, and depression.

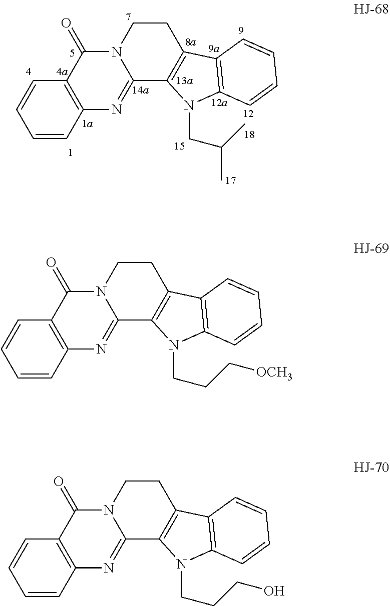 [SMILES: CC(C)Cn1c2c(c3ccccc31)CCn1c-2nc2ccccc2c1=O ; COCCCn1c2c(c3ccccc31)CCn1c-2nc2ccccc2c1=O ; O=c1c2ccccc2nc2n1CCc1c-2n(CCCO)c2ccccc12]

### TECHNICAL FIELD

The invention belongs to the field of separation of plant active ingredients and the field of medicine and health care. Specifically, the invention relates to an active ingredient of Zanthoxylum bungeanum Maxim and separation and use thereof, and in particular to an isoquinoline alkaloid compound and preparation method and use thereof.

### BACKGROUND OF THE ART

Alkaloids, which are a major class of active substances in plants of Zanthoxylum L., have analgesic and anti-inflammatory activities and are considered to be the characteristic components responsible for the unique tingling sensation. These alkaloids can be divided into four major categories according to their parent nuclei: quinoline derivatives, isoquinoline derivatives, benzophenanthridine derivatives and quinolone derivatives. Among them, isoquinoline alkaloids are characteristic ingredients owing to their significant pharmacological activities.

Voltage-gated potassium channels (Kv) have been discovered into 12 subfamilies, Kv1.x˜Kv12.x, with nearly 80 family members. Among them, Kv5, Kv6, Kv8, and Kv9 subfamily members alone do not form functional channels and are called silent Kv subunits (KvS). KvS channels can form functional heteromeric channels with Kv2.1 channels, regulating the current amplitude, activation and deactivation voltages, and dynamic characteristics of Kv2.1 channels. Current research believes that Kv2.1 channels are the main molecular basis for mediating delayed inward rectifier potassium currents (I_{K}) in primary tissues and cells such as dorsal root ganglion neuron (DRG), trigeminal ganglion neuron (TRG), pancreatic islets, hippocampus, and cardiovascular system. For example, in cultured small-diameter DRG neurons, Kv2.1 and Kv2.1/KvS channels accounts for about 60%, while Kv1.x and Kv3.x accounts for about 40% (Bocksteins, E. et al. Am J Physiol Cell Physiol. 296(6): C1271-8, 2009).

I_{K} potassium current and its main molecular basis Kv2.1 channels play important functions in normal physiological functions and disease states of the human body. The overexpression of Kv2.1 on the neuronal membrane surface leads to K⁺ efflux and aggravates neuronal death in the central Zn²⁺-induced post-stroke neuronal death pathway, and inhibiting Kv2.1 can be neuroprotective and exert therapeutic effects on stroke (Int J Mol Sci. 21(17):6107, 2020). In the cardiovascular system, I_{K} current mediates the repolarization of myocardial action potential and participates in the regulation of heart rhythm and blood pressure (Madeja, M. et al. J Biol Chem. 285(44): 33898-905, 2010). In pancreatic islets, elevated blood glucose levels stimulate depolarization of the membrane potential of pancreatic β-cells, which activates the Kv2.1 channels to hyperpolarize the membrane potential, reduce nerve excitability, and reduce insulin secretion. Inhibiting Kv2.1 can increase the duration and amplitude of action potentials in pancreatic beta cells, thereby increasing insulin secretion and reducing blood glucose levels (Jacobson, D. A. et al. Cell Metab. 6(3):229-35, 2007). IB4-negative small-diameter DRG neurons are mainly I_{K} currents which hyperpolarize the resting membrane potential, increase the amplitude and duration of the after hyperpolarization potential (AHP), increase the firing threshold, and prolong the action potential duration (Vydyanathan, A. et al. J Neurophysiol. 93(6): 3401-9, 2005). During the discharge process of central hippocampal neurons and trigeminal ganglion neurons under continuous stimulation, the regulatory effect of Kv2.1 on neural excitation depends on the duration of stimulation, and inhibiting Kv2.1 channels enhances the initial firing frequency but inhibits maintained firing (Liu P W et al. J Neurosci. 34(14): 4991-5002, 2014). Therefore, inhibiting Kv2.1 or I_{K} current can induce a neuroexcitatory desensitization effect similar to that induced by capsaicin and exert an analgesic effect (Arora, V. et al. Pharmacol Ther. 220:107743, 2021). Based on this, inhibiting I_{K} current or Kv2.1 channels can alleviate or treat diseases such as stroke, arrhythmia, diabetes and pain.

Two-pore domain potassium channels (K2P) underlie background (leak) K⁺ currents, play a key role in maintaining the resting membrane potential, and regulate the formation and firing of action potentials. Currently, 15 K2P channels have been cloned in mammals, and are divided into 6 subfamilies: TWIK, TREK, TASK, TALK, THIK, and TRESK. Hydroxy-α-sanshool isolated from Zanthoxylum bungeanum Maxim inhibits the TRESK (KCNK18) and TASK1/TASK3 (KCNK3/KCNK9) channels in the two-pore domain potassium channel DRG and TRG neurons respectively, promotes the firing of small-diameter and large-diameter neurons, and produces tingling (Bautista D M, et al. Nat Neurosci. 11(7): 772-9, 2008). In addition, antagonists of TRESK and TASK1/TASK3 channels are believed to have therapeutic effects on atrial fibrillation, respiratory depression, and depression (Mathie, A. et al., Annu Rev Pharmacol Toxicol. 61: 401-420, 2021).

A variety of active ingredients including isoquinoline derivatives have been isolated from Zanthoxylum bungeamum Maxim, but the action mechanism of these active ingredients is generally unclear. There is still a need for some active molecules with novel structures, clear mechanisms, and specific application types of diseases in this field.

### SUMMARY OF THE INVENTION

In the present invention, three isoquinoline alkaloid compounds, which are extracted from Zanthoxylum bungeamum Maxim and are not reported in literature, are discovered and disclosed for the first time. After extensive research, it is confirmed for the first time that these isoquinoline alkaloid compounds are inhibitors of I_{K} potassium current, especially Kv2.1 channels in the nervous system, cardiovascular system, and pancreas. In addition, these isoquinoline alkaloid compounds also have an inhibitory effect on TRESK potassium channel, and their activities are stronger than that of reported hydroxy-α-sanshool. Based on this, the present invention confirms that these isoquinoline alkaloid derivatives have analgesic activity in vivo by using a classic animal model of inflammatory pain.

Therefore, an object of the present invention is to provide an isoquinoline alkaloid compound.

Another object of the present invention is to provide a method for preparing the above-mentioned isoquinoline alkaloid compound.

Still another object of the present invention is to provide a use of the above-mentioned isoquinoline alkaloid compound.

In one aspect, the invention provides an isoquinoline alkaloid compound selected from the group consisting of the compounds represented by the following structural formulas:

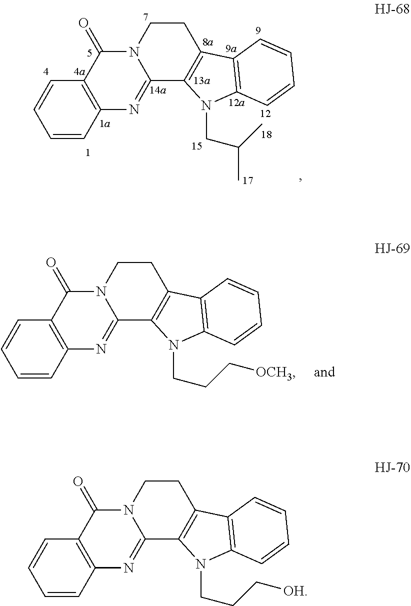 [SMILES: CC(C)Cn1c2c(c3ccccc31)CCn1c-2nc2ccccc2c1=O ; COCCCn1c2c(c3ccccc31)CCn1c-2nc2ccccc2c1=O ; O=c1c2ccccc2nc2n1CCc1c-2n(CCCO)c2ccccc12]

In another aspect, the invention provides a preparation method of the above-mentioned isoquinoline alkaloid compound, comprising the following steps:
(1) a dried Zanthoxylum bungeanum Maxim is pulverized and extracted with the solvent, and the resultant extract is concentrated to obtain a concentrated Zanthoxylum bungeamum Maxim extract;
(2) the concentrated Zanthoxylum bungeamum Maxim extract is dissolved in water and extracted with petroleum ether, ethyl acetate, and n-butanol respectively to obtain a petroleum ether phase, an ethyl acetate phase, a n-butanol phase, and a water phase;
(3) the ethyl acetate phase is subjected to gradient elution on a 200-300 mesh silica gel column by using mixtures of petroleum ether and acetone mixture at volume ratios of 50:1, 30:1, 20:1, 10:1, 8:1, 5:1, 2:1 and 1:1, respectively, as eluents to obtain six fractions of Fr. 1 to Fr. 6 in sequence; the Fr. 4 is subjected to gradient elution on a C18 reversed-phase silica gel column by using mixtures of water and methanol at volume ratios of 1:1, 1:2 and 1:4 in sequence as eluents to obtain eight fractions of Fr. 4-1 to Fr. 4-8 in sequence; the Fr. 4-6 is subjected to elution on a Megres C18 column by using mixtures of water and methanol as eluents, wherein HJ-68 is obtained by eluting with a mixture of water and methanol at 90% by volume and HJ-69 and HJ-70 are obtained by eluting with a mixture of water and methanol at 80% by volume.

In the above step (3), for example, the 6 fractions of Fr. 1 to Fr. 6 can be obtained as follows. The eluate is received in 500 ml beakers to obtain a series of eluates in sequence. The 500 mL solution in each beaker is evaporated to dryness and transferred to sample bottles. After complete elution of the ethyl acetate phase, the samples are spotted on a thin layer chromatography plate, and the samples having the same composition are combined as a same fraction, and thus 6 fractions of Fr. 1 to Fr. 6 are obtained in sequence. However, the present invention is not limited thereto.

In the above step (3), for example, the 8 fractions of Fr. 4-1 to Fr. 4-8 can be obtained as follows. The eluate is received in 50 mL graduated tubes to obtain a series of eluates in sequence. The 50 mL solution in each tube is evaporated to dryness and transferred to sample bottles. After elution is completed, the samples are spotted on a reversed-phase high-performance thin layer chromatography plate, and the samples having the same composition are combined as a same fraction, and thus 8 fractions of Fr. 4-1 to Fr. 4-8 are obtained in sequence. However, the present invention is not limited thereto.

The three compounds HJ-68, HJ-69 and HJ-70 isolated from Zanthoxylum bungeamum Maxim are all isoquinoline alkaloids with their structural formulas as follows:

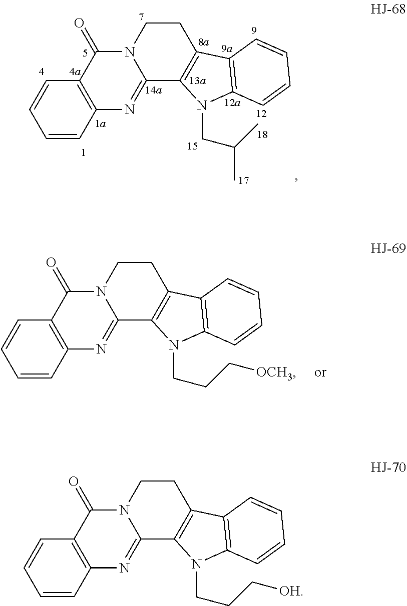 [SMILES: CC(C)Cn1c2c(c3ccccc31)CCn1c-2nc2ccccc2c1=O ; COCCCn1c2c(c3ccccc31)CCn1c-2nc2ccccc2c1=O ; O=c1c2ccccc2nc2n1CCc1c-2n(CCCO)c2ccccc12]

In step (1) of the preparation method of the present invention, the solvent may be one or a mixed solvent of two or more selected from the group consisting of water, methanol, ethanol, acetone and methylene chloride, and examples thereof include, but are not limited to, alcoholic solvents, for example, 70% ethanol in water, ethanol, 70% methanol in water, methanol, acetone, methylene chloride, or a combination thereof.

In step (1) of the preparation method of the present invention, the extraction may be a soaking extraction at room temperature, or an extraction under heating and refluxing. The extraction may be performed one or more times, preferably, by soaking in a solvent at room temperature 3 times, each time for 5-7 days, or by extraction under heating and refluxing 3 times, each time for 1-2 hours.

In step (3) of the preparation method of the present invention, preferably, the liquid phase separation conditions for HJ-68 are: mobile phase: methanol/water=90/10 (v/v); flow rate: 4 mL/min; column: Megres C18 column with length×diameter=250 mm×20 mm.

In step (3) of the preparation method of the present invention, preferably, the liquid phase separation conditions for HJ-69 are: mobile phase: methanol/water=80/20 (v/v); flow rate: 4 mL/min; column: Megres C18 column with length×diameter=250 mm×20 mm.

In step (3) of the preparation method of the present invention, preferably, the liquid phase separation conditions for HJ-70 are: mobile phase: methanol/water=80/20 (v/v); flow rate: 4 mL/min; column: Megres C18 column with length×diameter=250 mm×20 mm.

The present invention discloses and confirms for the first time that the above-mentioned isoquinoline alkaloid compounds selectively inhibit delayed inward rectifier potassium currents (I_{K}) of DRG neurons and has no significant effect on the sodium current and transient outward potassium current (I_{A}) of DRG neurons.

The present invention further confirms that the above-mentioned isoquinoline alkaloid compounds serve as an antagonist of the Kv2.1 potassium channel, which is the main molecular basis of I_{K} current, with an inhibitory activity equivalent to that for I_{K} current.

Therefore, in yet another aspect, the present invention provides a use of the above-mentioned isoquinoline alkaloid compound in preparation of an antagonist of delayed inward rectifier potassium current (I_{K}), especially, Kv2.1 channel.

Specifically, the present invention provides a use of the above-mentioned isoquinoline alkaloid compound as an antagonist of I_{K} current, especially, Kv2.1 channel. Therefore, the above-mentioned isoquinoline alkaloid compound can be used as a medicament, for example, for alleviating or treating a disease such as stroke, arrhythmia, diabetes, and pain, especially for alleviating or treating pain.

In addition, the present invention discloses also for the first time that the above-mentioned isoquinoline alkaloid compound is an antagonist of two-pore domain potassium (K2P) channel, especially TRESK channel. The present invention confirms that the above-mentioned isoquinoline alkaloid compound has an inhibitory activity on K2P channel which is stronger than that of hydroxy-α-sanshool that has been reported.

Therefore, in yet another aspect, the present invention provides a use of the above-mentioned isoquinoline alkaloid compound in preparation of an antagonist of K2P two-pore domain potassium (K2P) channel, especially TRESK channel.

Specifically, the present invention provides a use of the above-mentioned isoquinoline alkaloid compounds as an antagonist of two-pore domain potassium (K2P) channel, especially, TRESK channel. Therefore, the above-mentioned isoquinoline alkaloid compound can be used as a drug, for example, for alleviating or treating a disease such as atrial fibrillation, respiratory depression, and/or depression.

Therefore, in yet another aspect, the present invention provides a use of the above-mentioned isoquinoline alkaloid compound in the preparation of a medicament for treating a disease such as stroke, arrhythmia, diabetes, pain, atrial fibrillation, respiratory depression, and/or depression.

### BRIEF DESCRIPTION OF THE DRAWINGS

FIG. 1 shows the effects of HJ-70 on the spontaneous firing frequency in hippocampal neurons and evoked firing frequency in DRG neurons, wherein A shows the effect of 10 μM HJ-70 on the spontaneous firing frequency in hippocampal neurons; B is a statistical diagram of the effect of 10 μM HJ-70 on the spontaneous firing frequency in hippocampal neurons; C shows the effect of 10 μM HJ-70 on the evoked firing frequency in DRG neurons; and D is a statistical diagram of the effect of 10 μM HJ-70 on the evoked firing frequency in DRG neurons. ***P≤0.001.

FIG. 2 shows the effects of HJ-70 on the potassium currents in DRG neurons, wherein A shows the potassium currents recorded from the DRG neurons under control condition; B shows the effect of 3 μM HJ-70 on the potassium currents in DRG neurons; C shows the effect of 10 μM HJ-70 on the potassium currents in DRG neurons; D shows the potassium currents in DRG neurons after washout of the HJ-70; E is a statistical diagram of the potassium currents in DRG neurons inhibited with 3 μM HJ-70; F is a statistical diagram of the potassium currents in DRG neurons inhibited with 10 μM HJ-70; G is a statistical diagram of the potassium currents in DRG neurons after washout of the HJ-70.** represents P≤0.01,*** represents P≤0.001.

FIG. 3 shows the effects of HJ-70 on the potassium currents in hippocampal neurons, wherein A shows the effect of 1 μM HJ-70 on the potassium currents in hippocampal neurons; B shows the effect of 3 μM HJ-70 on the potassium currents in hippocampal neurons; C shows the effect of 10 μM HJ-70 on the potassium currents in hippocampal neurons; D shows the effect of 5 mM TEA as the positive drug on the potassium currents in hippocampal neurons; E is a statistical diagram of the effect of 1 μM HJ-70 on the potassium currents in hippocampal neurons; F is a statistical diagram of the effect of 3 μM HJ-70 on the potassium currents in hippocampal neurons; G is a statistical diagram of the effect of 10 μM HJ-70 on the potassium currents in hippocampal neurons; and H is a statistical diagram of the effect of 5 mM TEA as the positive drug on the potassium currents in hippocampal neurons. The ns represents no statistical difference, ** represents P≤0.01, and*** represents P≤0.001.

FIG. 4 shows the effects of HJ-70 on the sodium currents in hippocampal neurons with the membrane potential clamped at −90 mV, wherein A shows the effect of 10 μM HJ-70 on the sodium currents in hippocampal neurons; and B is a statistical diagram of the inhibitory effect of 10 μM HJ-70 on the sodium currents in hippocampal neurons.*** represents P≤0.001.

FIG. 5 shows the effects of HJ-70 on the voltage-gated potassium channels transiently expressed in CHO cells, wherein A shows the effect of 10 μM HJ-70 on Kv2.1 channel; B is a statistical diagram of the effect of 10 μM HJ-70 on Kv2.1 channel; C shows the effect of 10 μM HJ-70 on TRESK channel; and D is a statistical diagram of the effect of 10 μM HJ-70 on TRESK channel.*** represents P≤0.001.

FIG. 6 shows the dose-dependent effects of HJ-70 on formaldehyde-induced inflammatory pain behavior, wherein A shows the alleviating effects of 100 mg/kg HJ-70 on pain behaviors in formalin model; B shows the alleviating effect of 50 mg/kg HJ-70 on pain behaviors in formalin model; C shows the alleviating effect of 300 mg/kg HJ-70 on pain behaviors in formalin model; D is a statistical diagram of the licking time and pain scores of inflammatory pain in the first phase (0-10 min) of the formalin model; and E is a statistical diagram of the licking time and pain scores of inflammatory pain in the second phase (10-60 min) of the formalin model. * represents P≤0.05,** represents P≤0.01, and*** represents P≤0.001.

### DETAILED DESCRIPTION OF EXEMPLARY EMBODIMENTS

The content and beneficial effects of the present invention will be further described below with reference to examples, which are only used to illustrate the present invention without any limitation.

### EXAMPLES

The experimental methods used in the following examples are conventional methods unless otherwise specified.

All of the materials, reagents, etc. used in the following examples can be obtained from commercial sources unless otherwise specified.

Sample to be extracted: Zanthoxylum bungeamum Maxim.

Solvent for extraction, isolation and purification: ethanol, water, petroleum ether, ethyl acetate, n-butanol.

Instruments: extraction tank (self-made), Tokyo Rika 20 L rotary evaporator (Tokyo Rika, Japan), R-210 rotary evaporator (BUCHI Company, Switzerland), analytical high performance liquid chromatograph with a maximum flow rate of 10 mL (Jiangsu Hanbang Technology Co., Ltd.), Bruker Avance III-400 nuclear magnetic resonance instrument (Bruker Company, Germany), Bruker microTOF-Q II high-resolution mass spectrometer (Bruker Company, Germany), IFS120 HR 670 FT-IR infrared spectrometer (Bruker Company, Germany) Lambda 35 UV-visible spectrophotometer (Perkin Elmer Company, USA).

### Example 1: Extraction Method of Active Ingredients of Zanthoxylum Bungeanum Maxim

1.1 A dried Zanthoxylum bungeamum Maxim was pulverized and extracted three times by soaking in 10 times by mass of 70% ethanol at room temperature, each time for 5-7 days. The combined extract was concentrated until there was no alcohol smell, to obtain a concentrated extract of Zanthoxylum bungeanum Maxim.

The concentrated extract of Zanthoxylum bungeamum Maxim was dissolved in water, and extracted with petroleum ether, ethyl acetate, and n-butanol respectively to obtain a petroleum ether phase, an ethyl acetate phase, a n-butanol phase, and a water phase.

The ethyl acetate phase was separated on a 200-300 mesh silica gel column by gradient elution with mixtures of petroleum ether and acetone (the volume ratios of petroleum ether:acetone in the mixtures were 50:1, 30:1, 20:1, 10:1, 8:1, 5:1, 2:1, and 1:1 respectively). The eluate was received in 500 mL beakers, and the 500 mL solution in each beaker was evaporated to dryness and transferred to sample bottles. After complete elution of the ethyl acetate phase, the samples were spotted on a thin layer chromatography plate and the samples having the same composition were combined as a same fraction, and 6 fractions of Fr. 1 to Fr. 6 were obtained in total.

Among them, Fr. 4 was separated on a C18 reverse-phase silica gel column by gradient elution with mixtures of water and methanol at water:methanol volume ratios of 1:1, 1:2, and 1:4 in sequence. Finally, the column was flushed with methanol. The eluate was received in 50 mL graduated tubes. The 50 mL solution in each tube was evaporated to dryness and transferred to sample bottles. After the elution was completed, the sample was spotted on a reversed-phase high-performance thin-layer chromatography plate. The samples having the same composition were combined as a same fraction, and 8 fractions of Fr. 4-1 to Fr. 4-8 were obtained in total.

Fr. 4-6 was separated on a Megres C18 10 mm×250 mm analytical liquid phase chromatographic column with a maximum flow rate of 10 mL/min and a mobile phase flow rate of 4 mL/min to obtain HJ-68 (the mobile phase was 90% by volume methanol in water, and the peak time was 5.4 min); HJ-69 (the mobile phase was 80% by volume methanol in water, the peak time was 10.1 min); HJ-70 (the mobile phase was 80% by volume methanol in water, the peak time was 12.0 min).

The experimental data of N-13-isobutylrutaecarpine (HJ-68): yellow needle-shaped crystal; IR (film) νₘₐₓ 2926, 255 2858, 1728, 1669, 1637, 1587, 1465, 1292, 1130 cm⁻¹; UV(CH₃OH) λₘₐₓ (log ε) 216 (3.06), 234 (2.99), 329 (2.99), 345 (3.05), 362 (2.95) nm; See Table 1 for ¹H and ¹³C NMR; HRESIMS m/z: 344.1757 calculated for C₂₂H₂₂N₃O, Found: 344.1764 [M+H]⁺.

The experimental data of N-13-methoxypropylrutaecarpine (HJ-69): yellow needle-shaped crystal; IR (film) νₘₐₓ 2924, 2872, 1662, 1585, 1534, 1465, 1332, 1203, 1114 cm⁻¹; UV(CH₃OH) λₘₐₓ (log ε) 215 (3.34), 235 (3.28), 328 (3.28), 344 (3.44), 362 (3.24) nm; See Table 1 for ¹H and ¹³C NMR; HRESIMS m/z: 382.1526 calculated for C₂₂H₂₁N₃O₂Na, Found: 382.1530 [M+Na]⁺.

The experimental data of compound N-13-n-propanol rutaecarpine (HJ-70): yellow needle-shaped crystal; IR (film) νₘₐₓ 3430, 2925, 2857, 1729, 1665, 1586, 1466, 1291, 1048 cm⁻¹; UV(CH₃OH) λₘₐₓ (log ε) 216 (3.33), 235 (3.26), 329 (3.24), 344 (3.29), 361 (3.18) nm; See Table 1 for ¹H NMR (400 M Hz) and ¹³C NMR (100 M Hz); HRESIMS m/z: 346.1550 calculated for C₂₁H₂₀N₃O₂, Found: 346.1542 [M+H]⁺.

TABLE 1
¹H and ¹³C NMR values of HJ-68, HJ-69 and HJ-70 (the deuterated reagent is CDCl₃).

| | HJ-68 | | HJ-69 | | HJ-70 | |
|---|---|---|---|---|---|---|
| no | ¹H (J in Hz) | ¹³C, type | ¹H (J in Hz) | ¹³C, type | ¹H (J in Hz) | ¹³C, type |
| 1 | 7.67 d (8.0) | 127.2, d | 7.66 d (8.0) | 127.2, d | 7.71 d (8.0) | 126.8, d |
| 2 | 7.72 t (8.1) | 134.2, d | 7.70 t (8.1) | 134.1, d | 7.72 t (7.9) | 134.7, d |
| 3 | 7.41 t (8.1) | 126.2, d | 7.39 t (8.1) | 126.2, d | 7.48 t (8.3) | 126.8, d |
| 4 | 8.31 d (7.9) | 127.1, d | 8.29 d (8.0) | 127.0, d | 8.30 d (8.0) | 127.3, d |
| 4a | | 120.9, s | | 120.9, s | | 120.8, s |
| 1a | | 147.4, s | | 147.4, s | | 146.5, s |
| 5 | | 161.8, s | | 161.7, s | | 161.5, s |
| 7 | 4.54 t (2H, 6.8) | 41.0, d | 4.49 t (2H, 6.8) | 40.9, d | 4.52 t (2H, 6.8) | 41.1, d |
| 8 | 3.17 t (2H, 6.8) | 19.8, d | 3.14 t (2H, 6.5) | 19.8, d | 3.18 t (2H, 6.7) | 19.8, d |
| 8a | | 119.3, s | | 119.2, s | | 120.4, s |
| 9a | | 124.1, s | | 124.0, s | | 124.5, s |
| 9 | 7.62 d (8.0) | 120.0, d | 7.60 d (8.0) | 119.9, d | 7.65 d (8.0) | 120.4, d |
| 10 | 7.16 t (7.2) | 120.2, d | 7.16 t (7.5) | 120.3, d | 7.21 t (7.3) | 120.8, d |
| 11 | 7.35 t (8.2) | 125.1, d | 7.39 t (8.6) | 125.2, d | 7.44 t (8.2) | 124.5, d |
| 12 | 7.43 d (8.4) | 111.1, d | 7.49 d (8.4) | 110.6, d | 7.48 d (8.5) | 111.2, d |
| 12a | | 140.5, s | | 140.2, s | | 140.1, s |
| 13a | | 126.8, s | | 126.2, s | | 126.8, s |
| 14a | | 145.5, s | | 145.2, s | | 146.1, s |
| 15 | 4.69 d (2H, 7.2) | 52.2, t | 4.90 t (2H, 7.1) | 42.3, t | 4.95 t (2H, 6.2) | 41.8, t |
| 16 | 2.41 m | 30.2, d | 2.21 m | 30.9, t | 2.28 m | 32.6, t |
| 17 | 0.96 d (3H, 6.8) | 20.4, q | 3.40 t (2H, 5.8) | 69.9, t | 3.61 t (2H, 5.3) | 58.3, t |
| 18 | 0.96 d (3H, 6.8) | 20.4, q | 3.31 s | 58.7, q | | |

### Example 2: Effects of HJ-70 on Action Potentials and Potassium Currents in Dorsal Root Ganglion Neurons

### 2.1 Isolation and Culture of Dorsal Root Ganglion Neurons:

A 4- to 6-week old C57BL/6 mouse (Shanghai Slack Experimental Animal Co., Ltd.) was used for acute isolation of dorsal root ganglion neurons. First, the mouse was sacrificed by breaking the neck, and the surface skin was disinfected with 75% alcohol. The skin was cut from the neck to the buttocks, and the spine from the neck to the waist was cut, and placed in a pre-cooled phosphate buffer (PBS) (Hyclone Company) after excess muscles and blood clots were removed. The spine was cut longitudinally along the midline, and the white spinal cord and blood clots in the middle were removed by ophthalmic tweezers to expose the intervertebral foramen. The dorsal root ganglia were taken out by fine forceps, wherein the large ganglia at the waist should be stripped of their fibrous connections. Then the ganglia were cut into small pieces with microdissection scissors, added to 1 mL of a mixture of collagenase (1 mg/mL) and trypsin (0.25 mg/mL) (Sigma-Aldrich Company) for digestion for 15 min. Then a DMEM/F12 culture medium (Gibco Company) supplemented with 10% fetal calf serum was added thereto to terminate the digestion and dilute the resultant. The cells were pipetted repeatedly, and large tissue blocks were filtered out with a 70 μM cell filter. The remaining liquid was inoculated into a glass slide pre-coated with poly-lysine (Sigma-Aldrich), and cultured in an incubator at 5% CO₂ and 37° C.

### 2.2 Recording of Action Potentials and Potassium Currents in Dorsal Root Ganglion Neurons:

Whole-cell patch clamp recording was performed through a digital-to-analog converter, the Digidata 1440A (Axon CNS), and a patch clamp amplifier, the Axon patch 700B (Molecular Devices), and the borosilicate glass electrode used in the manual patch clamp experiment was pulled by a Sutter-P1000 electrode puller (Sutter) with a resistance about 3 MΩ. The bath solution was continuously perfused in speed of about 2 mL/min using a BPS perfusion system.

The action potential recording protocol was as follows. The cells were clamped at 0 pA and injected with a current stimulation having an intensity of 200 pA and a duration of 500 ms to record the evoked action potential firing.

### Electrophysiological Recordings of Neuronal Potassium Currents:

The cell were clamped at a holding voltage of −50 mV, hyperpolarized to −110 mV, and then directly depolarized to 40 mV after 600 ms to record the whole cell potassium current (I_{Total}). To record the delayed inward rectifier potassium current (I_{K}), the cells were first depolarized to −50 mV, maintained for 50 ms, and then depolarized to 40 mV. 10 μM HJ-70 was administered by perfusion to detect drug efficacy, and 5 mM TEA was administered as a positive control.

TABLE 2
The external solution for electrophysiological
recording of neuronal potassium current

| Chemical | M.W. (g/mol) | C (mM) |
|---|---|---|
| NaCl | 58.44 | 140 |
| CaCl₂ | 110.99 | 1 |
| KCl | 74.56 | 5 |
| MgCl₂ | 203.30 | 1.25 |
| HEPES | 238.30 | 10 |
| Glucose | 180.16 | 10 |

The solution was adjusted to pH=7.4 with 1M KOH and stored at 4° C. for later use.

TABLE 3
The internal solution for electrophysiological
recording of neuron potassium current

| Chemical | M.W. (g/mol) | C (mM) |
|---|---|---|
| KCl | 74.55 | 140 |
| HEPES | 238.30 | 10 |
| EGTA | 380.35 | 10 |
| CaCl₂ | 110.99 | 1 |
| MgCl₂ | 203.30 | 1 |

The solution was adjusted to pH=7.2 with 1M KOH, filtered and packaged, and stored at 4° C. for later use.

### 2.3 Acute Isolation of Hippocampal Neurons:

9 mg of protease (type XXIII, Sigma) was weighed in advance, 5 mg of bovine serum albumin (BSA, Shanghai Sangon) and 5 mg of trypsin inhibitor (Sigma-Aldrich) were weighed, and 3 mg/mL and 5 mg/mL enzyme and termination solutions were respectively prepared with a dissociation solution, and incubated at 32° C. water bath. 2 SD rats that were 1-7 days old (Shanghai Slack Experimental Animal Co., Ltd.) were decapitated and cut the cortex and skull to take out the complete brain, which was divided into two parts along the midline of the brain, and the hippocampus was located in the temporal lobe of the brain. The temporal lobe cortex was removed to expose the hippocampal gyrus for dissection. Then the hippocampus tissue was slightly moistened with the dissociation solution, and cut into thin slices transversely with a razor blade. The slices were moved into a digestive solution for digestion for 8 minutes, and then the digestive solution was poured off. The termination solution saturated with oxygen was added and incubated for 1-2 hours. 3-4 seahorse-shaped slices were put in the dissociation solution and pipetted into a suspension and settled for a while to precipitate large tissue pieces. The suspension was dropped into a prepared dish and allowed to stand for a few minutes. Half of the dissociation solution was replaced with the extracellular solution, and then patch-clamp experiments on potassium and sodium channels in neurons were conducted by rapid administration.

### 2.4 Primary Culture of Hippocampal Neurons:

Enzyme and termination solutions with the same concentration were prepared with PBS. 90% DMEM/F12 supplemented with 10% FBS, neuron culture medium (2% B-27, 1% penicillin and streptomycin mixture, Neurobasal-A supplemented with 0.5 mM 1% GlutaMAX), and enzyme and termination solutions (all purchased from Gibco) were preheated in advance. The slides were coated in a 24-well plate in advance with 20 μg/mL polylysine. All operations were performed in a biological safety cabinet. 3 newly born SD rats that were 1-3 days old were taken out the hippocampus according to the above method. The hippocampus was cut into pieces with microdissecting scissors and digested for 8 minutes in the enzyme solution, which was slightly shaken every 2-3 minutes to make the digestion even. Then the enzyme solution was aspirated out, and the termination solution was added to terminate the reaction. The hippocampus was gently pipetted to form a suspension, which was filtered through a 70 μM filter. The filtrate was centrifuged at 1100 rpm for 5 minutes. The supernatant was discarded, and 5 mL of a culture medium was added to re-suspend the cells. The suspension was counted on a cell counter, and adjusted to a final concentration of 10⁵ cells/mL. The suspension was then placed in a carbon dioxide incubator for culture. After 6 hours, the medium was completely replaced with the neuron culture medium. Then half of the medium was replaced every 3 days. After 14-day incubation, electrophysiological recordings of spontaneous neuronal firing were performed by perfusion administration.

TABLE 4
The external solution for electrophysiological
recording of neuronal sodium current

| Chemical | M.W. (g/mol) | C (mM) |
|---|---|---|
| NaCl | 58.44 | 120 |
| CaCl₂ | 110.99 | 1 |
| TEA-Cl | 165.70 | 20 |
| KCl | 74.56 | 5 |
| MgCl₂ | 203.30 | 1.25 |
| HEPES | 238.30 | 10 |
| Glucose | 180.16 | 10 |

The solution was adjusted to pH=7.4 with 1M NaOH, and stored at 4° C. after oxygen saturation for later use.

TABLE 5
The internal solution for electrophysiological
recording of neuronal sodium current

| Chemical | M.W. (g/mol) | C (mM) |
|---|---|---|
| CsCl | 168.36 | 120 |
| NaCl | 58.44 | 10 |
| TEA-Cl | 165.70 | 10 |
| HEPES | 238.30 | 10 |
| EGTA | 380.35 | 10 |
| CaCl₂ | 110.99 | 1 |
| MgCl₂ | 203.30 | 1 |

The solution was adjusted to pH=7.2 with 1M CsOH, filtered and packaged, and stored at 4° C. for later use.

### 2.5 Analysis of Experimental Results:

The data were processed through Clampfit 10.2, and imported into an Excel table for statistics, and then sorted and analyzed for their significant differences through GraphPad Prim5 software. The transient outward potassium current (I_{A}) was obtained by subtracting the delayed inward rectifier potassium current from the total potassium current. All electrophysiological experiment data were expressed as mean±standard error (mean±S.E.M.). The significant difference comparison of the data was performed using Student's paired t-test. *P≤0.05,**P≤0.01, ***P≤0.001 indicated that there is a significant difference between the two groups and is statistically significant.

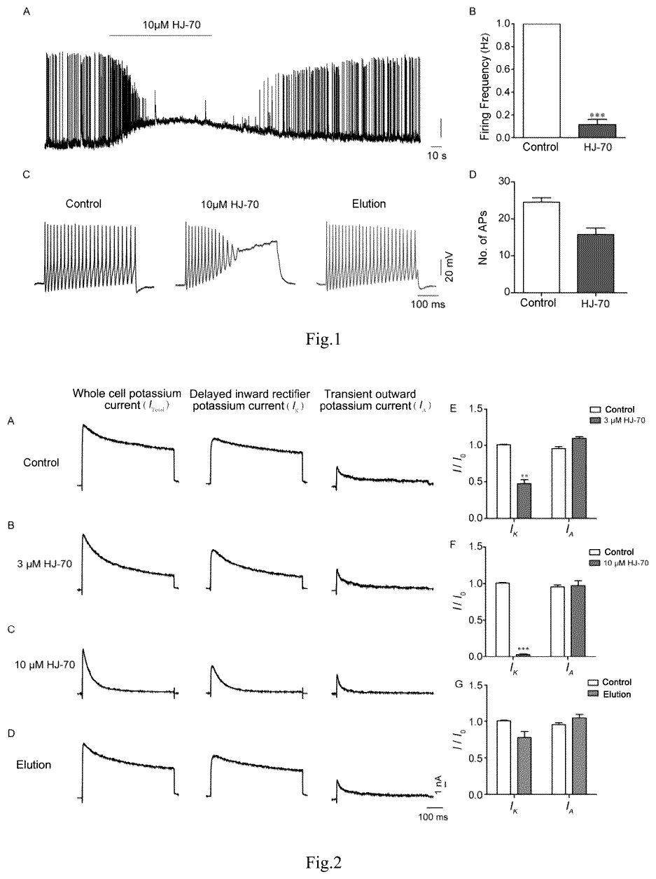

It can be shown from the results that 10 μM HJ-70 could significantly inhibit the spontaneous firing of cultured hippocampal neurons (A and B in FIG. 1). In addition, 10 μM HJ-70 also significantly inhibited the evoked action potential firing frequency of dorsal root ganglion (DRG) (C and D in FIG. 1). Therefore, HJ-70 could inhibit central and peripheral neuronal excitability.

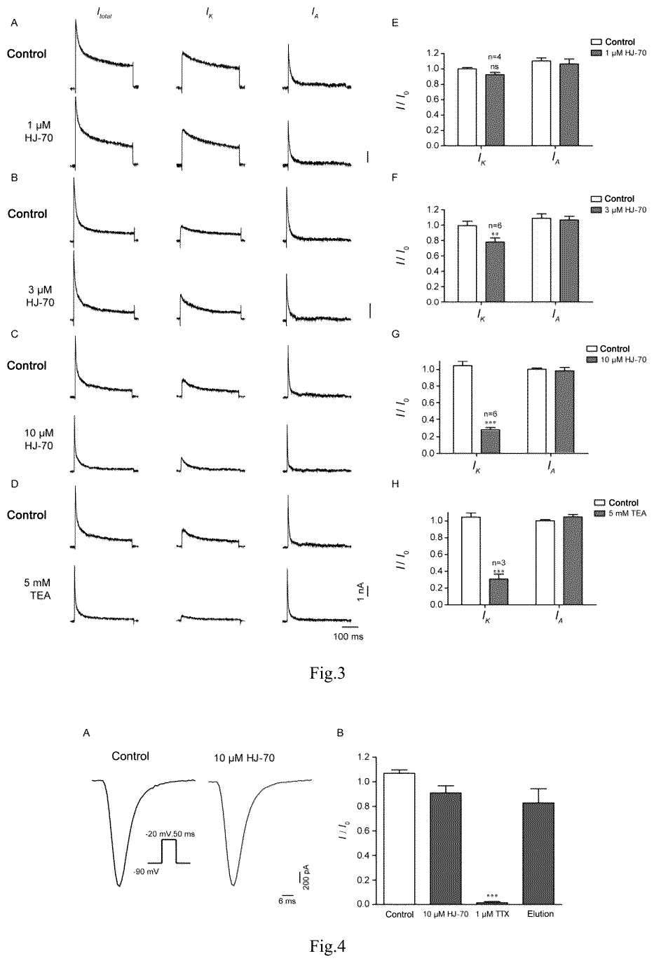

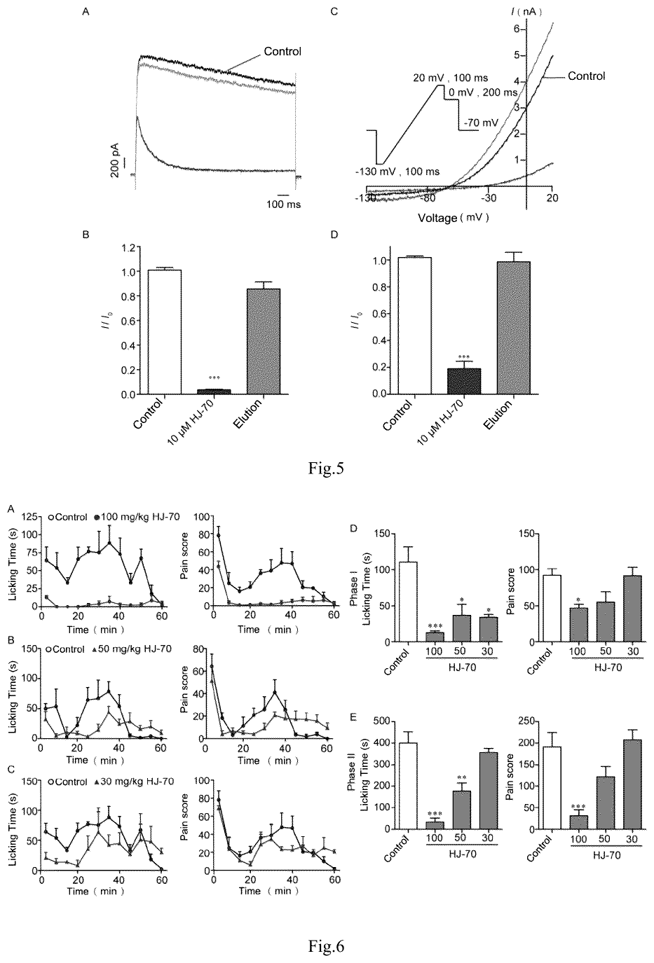

Sodium and potassium currents are the classic molecular basis for mediating neuronal firing. In view of the fact that HJ-70 inhibits neuronal electrical excitability, the effects of HJ-70 on potassium and sodium currents in DRG and hippocampal neurons were further examined. As shown in B and C in FIG. 2, HJ-70 dose-dependently inhibited the I_{K} current in the DRG but had no significant effect on the I_{A} current. It is worth noting that the inhibitory effect on Iₖ current caused by HJ-70 can be completely eluted (G in FIG. 2), indicating the reversible inhibition of I_{K} current by HJ-70. Similar as the inhibition on the activity of DRG neurons in the peripheral nervous system, HJ-70 reversibly and dose-dependently inhibited the I_{K} current of hippocampal neurons without affecting the I_{A} current (FIG. 3).

The effect of HJ-70 on sodium current in hippocampal neurons was further examined, and it was found that 10 μM HJ-70 had no effect on sodium current in hippocampal neurons (A in FIG. 3), while 1 μM sodium channel selective inhibitor could completely inhibit sodium current (B in FIG. 3).

In summary, HJ-70 dose-dependently inhibited I_{K} current in neurons without affecting I_{A} potassium current and sodium current, thereby reducing the electrical excitability of hippocampal neurons and DRG neurons.

### Example 3: HJ-70 Inhibited the Currents of Kv2.1 and TRESK Channels

### 3.1 Electrophysiological Recordings:

First, TRESK plasmid (a kind gift from Professor Min LI of Johns Hopkins University) and Kv2.1 plasmid (a kind gift from Professor Min LI of Johns Hopkins University) were each co-transfected into CHO-K1 cells (ATCC) with green fluorescent protein EGFP plasmid (a kind gift from Professor Jia LI of SHANGHAI INSTITUTE OF MATERIA MEDICA, CHINESE ACADEMY OF SCIENCES) at a ratio of 9:1, and the cells with green fluorescence were selected under a fluorescence microscope to perform the experiments. The cells were clamped at −70 m V, first hyperpolarized to −130 mV, given a ramp depolarization stimulus to 20 mV, maintained for 100 ms, and then hyperpolarized to 0 mV to record the potassium channel current. 10 μM HJ-70 was administered by perfusion to test the drug efficacy thereof.

### 3.2 Data Analysis:

Current researches show that Kv2.1 channel is the main molecular basis for mediating Iₖ currents (see the section of BACKGROUND OF THE ART for details). In view of the dose-dependent inhibition of I_{K} current by HJ-70, the effect on Kv2.1 currents was further examined. As shown in A and B in FIG. 5, 10 μM HJ-70 completely inhibited Kv2.1 channel-mediated currents, which is consistent with the activity in neurons. In addition, hydroxy-α-sanshool, another active ingredient in Zanthoxylum bungeanum Maxim, inhibited TRESK and TASK1/TASK3 channels in neurons with a half-effective dose of about 50 μM. Therefore, the effect of HJ-70 on TRESK channel was evaluated, and it was found that 10 μM HJ-70 almost completely inhibited the TRESK channel current (C and D in FIG. 5), indicating that the inhibitory activity of HJ-70 on TRESK is stronger than that of hydroxy-α-sanshool. In summary, this result indicated that HJ-70 is an inhibitor of Kv2.1 and TRESK potassium channels.

### Example 4: HJ-70 Significantly Alleviated Formaldehyde-Induced Inflammatory Pain

### 4.1 Animal Experiment:

A 10% formaldehyde solution was diluted 10 times to prepare a 1% formaldehyde solution. 5% DMSO, 5% Tween-80 and 90% normal saline (a 0.9% NaCl solution) were used to prepare solutions of HJ-70 in 100 mg/kg and 30 mg/kg. Each mouse was administered at a dose of 0.2 mL/10 g at 30 minutes before the experiment, and then 20 μL of 1% formaldehyde solution was injected into the sole of the foot through a microsampling needle to create a formalin model. The number of licking of hind paws, flicking of hind paws, and paw lifting, and the time of paw licking and prolonged paw lifting were recorded within 60 minutes. Paw licking was scored as 3, paw flicking was scored as 2, and paw lifting was scored as 1. The pain behavior score and time were calculated to determine the analgesic activity of HJ-70.

### 4.2 Analysis of Experimental Results:

Data sorting and statistics were performed in the same way as electrophysiological data processing, and then One-Way ANOVA was used for analysis in GraphPad Prism 5 software.*P<0.05,**P<0.01,***P<0.001 indicated that there is a significant difference between the two groups and is statistically significant.

In view of the important role of I_{K} potassium current and background potassium current, especially Kv2.1 and TRESK channels, in stroke, arrhythmia, diabetes, pain, atrial fibrillation, respiratory depression and/or depression (see section of BACKGROUND OF THE ART for details), the effect of HJ-70 on pain was evaluated. The formalin-induced mouse pain model is a classic animal model of inflammatory pain and is widely used in analgesic drug discovery and research. Consistent with the in vitro experimental results, it was found that intraperitoneal injection of HJ-70 could dose-dependently alleviate the pain behavioral response induced by plantar injection of formalin in mice (D and E in FIG. 5). This result shows that HJ-70 has clear in vivo activity.

1. An isoquinoline alkaloid compound selected from the group consisting of the compounds represented by the following structural formulas:

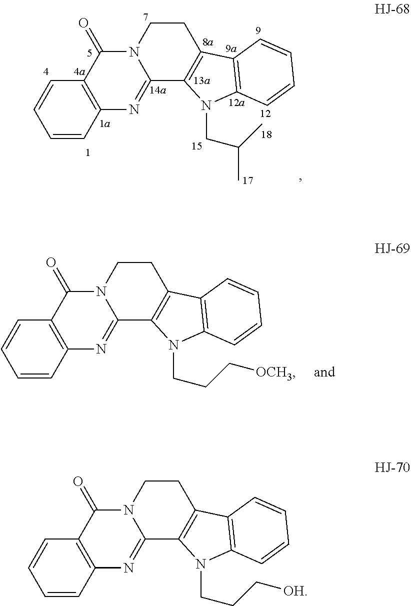 [SMILES: CC(C)Cn1c2c(c3ccccc31)CCn1c-2nc2ccccc2c1=O ; COCCCn1c2c(c3ccccc31)CCn1c-2nc2ccccc2c1=O ; O=c1c2ccccc2nc2n1CCc1c-2n(CCCO)c2ccccc12]

2. A preparation method of the isoquinoline alkaloid compound according to claim 1, comprising the following steps:
(1) a dried Zanthoxylum bungeamum Maxim is pulverized and extracted with the solvent, and the resultant extract is concentrated to obtain a concentrated Zanthoxylum bungeamum Maxim extract;
(2) the concentrated Zanthoxylum bungeamum Maxim extract is dissolved in water and extracted with petroleum ether, ethyl acetate, and n-butanol respectively to obtain a petroleum ether phase, an ethyl acetate phase, a n-butanol phase, and an aqueous phase;
(3) the ethyl acetate phase is subjected to gradient elution on a 200-300 mesh silica gel column by using mixtures of petroleum ether and acetone at volume ratios of 50:1, 30:1, 20:1, 10:1, 8:1, 5:1, 2:1 and 1:1, respectively, as eluents to obtain six fractions of Fr. 1 to Fr. 6 in sequence; the Fr. 4 is subjected to gradient elution on a C18 reversed-phase silica gel column by using mixtures of water and methanol at volume ratios of 1:1, 1:2 and 1:4 in sequence, as eluents to obtain eight fractions of Fr. 4-1 to Fr. 4-8 in sequence; the Fr. 4-6 is subjected to elution on a Megres C18 column by using mixtures of water and methanol as eluents, wherein HJ-68 is obtained by eluting with a mixture of water and methanol at 90% by volume and HJ-69 and HJ-70 are obtained by eluting with a mixture of methanol and water at 80% by volume.

3. The preparation method according to claim 2, wherein in step (1), the solvent is one or a mixed solvent of two or more selected from the group consisting of water, methanol, ethanol, acetone and methylene chloride, preferably the solvent is 70% by volume ethanol in water, ethanol, 70% by volume methanol in water, methanol, acetone, methylene chloride, or a combination thereof;
wherein in step (1), the extraction is a soaking extraction at room temperature or an extraction under heating and refluxing, preferably, the extraction is performed one or more times, more preferably, the extraction is performed by soaking with a solvent at room temperature 3 times, each time for 5-7 days, or the extraction is performed by extraction under heating and refluxing 3 times, each time for 1-2 hours.

4. The preparation method according to claim 2, wherein in step (3), the liquid phase separation conditions for HJ-68 are as follows:
the mobile phase is methanol/water at a volume ratio of 90/10, the flow rate is 4 mL/min; and the column is Megres C18 column with length×diameter of 250 mm×20 mm.

5. The preparation method according to claim 2, wherein in step (3), the liquid phase separation conditions for HJ-69 are as follows:
the mobile phase is methanol/water at a volume ratio of 80/20, the flow rate is 4 mL/min; and the column is Megres C18 column with length×diameter of 250 mm×20 mm.

6. The preparation method according to claim 2, wherein in step (3), the liquid phase separation conditions for HJ-70 are as follows:
the mobile phase is methanol/water at a volume ratio of 80/20, the flow rate is 4 mL/min; and the column is Megres C18 column with length×diameter of 250 mm×20 mm.

7. Use of the isoquinoline alkaloid compound according to claim 1 in preparation of an antagonist for delayed inward rectifier potassium current (I_{K}), in particular, Kv2.1 channel.

8. Use of the isoquinoline alkaloid compound according to claim 1 in preparation of an antagonist for two-pore domain potassium (K2P) channel, especially TRESK channel.

9. Use of the isoquinoline alkaloid compound according to claim 1 in preparation of a medicament for treating stroke, arrhythmia, diabetes, pain, atrial fibrillation, respiratory depression, and/or depression.
